(19) United States
(12) Patent Application Publication
(10) Pub. No.: US 2020/0338223 A1
(43) Pub. Date: Oct. 29, 2020

(54) METHOD OF PRODUCING MICROBIAL FERMENTATION BROTH

(71) Applicant: IZANA HOLDINGS. Co., LTD., SEOUL (KR)

(72) Inventor: Hong Suk CHAE, Seoul (KR)

(21) Appl. No.: 16/670,236

(22) Filed: Oct. 31, 2019

(30) Foreign Application Priority Data
Apr. 25, 2019 (KR) 10-2019-0048456

(51) Int. Cl. A61L 9/013; A61L 9/012; A61L 9/14; C12N 1/20; C12R 1/245; C12R 1/125; C12R 1/67; C12R 1/69; C12N 5/00

(52) U.S. Cl. CPC A61L 9/013; A61L 9/012; A61L 9/14; C12N 1/20; A61L 2209/21; C12R 1/125; C12R 1/67; C12R 1/69; C12N 5/0025; C12R 1/245

(57) ABSTRACT

A method of producing a microbial fermentation broth includes fermenting at least two of plum, dandelion, pine needle, and thistle to prepare each fermentation stock solution, mixing the fermentation stock solutions and fermenting the mixed fermentation stock solutions a to prepare a fermentation mixture, mixing the fermentation mixture with coffee meal and fermenting the mixed fermentation mixture with the coffee meal to prepare a coffee meal fermented solution, and aging the coffee meal fermented solution.

### CROSS-REFERENCE TO RELATED APPLICATIONS

A claim for priority under 35 U.S.C. § 119 is made to Korean Patent Application No. 10-2019-0048456 filed on Apr. 25, 2019, in the Korean Intellectual Property Office, the entire contents of which are hereby incorporated by reference.

### INCORPORATION OF SEQUENCE LISTING

This application contains a sequence listing submitted in Computer Readable Form (CRF). The CFR file containing the sequence listing is entitled “9-PK3593508-SequenceListing.txt”, which was created and modified on Oct. 30, 2019, and is 1,147 bytes in size. The information in the sequence listing is incorporated herein by reference in its entirety.

### BACKGROUND

Embodiments of the inventive concept described herein relate to a method of producing a microbial fermentation broth, and more particularly, relate to a method of producing a microbial fermentation broth, which includes microorganisms cultured by fermenting coffee meal.

As food waste and livestock manure are composted and spread to farmhouses, odors and poisonous gases are produced and thereby the farmhouses are severely damaged.

The odors from the food waste cause civil complaints to make local governments troubled. There is no specific solution, huge costs are incurred, and burden is increased.

Furthermore, when the food waste is buried, a bad smell, pests, and pathogenic bacteria are generated in landfills. In addition, soil and water are polluted and land devastation is serious.

### SUMMARY

Embodiments of the inventive concept provide a method of producing a microbial fermentation broth, where an extract of the microbial fermentation broth removes an odor and a by-product of the microbial fermentation broth is used as compost.

According to an exemplary embodiment, a method of producing a microbial fermentation broth includes fermenting at least two of plum, dandelion, pine needle, and thistle at 20° C. to 40° C. for 4 months to 8 months to prepare each fermentation stock solution, mixing the fermentation stock solutions and fermenting the mixed fermentation stock solutions at 20° C. to 30° C. for 1 month to 5 months to prepare a fermentation mixture, mixing the fermentation mixture with coffee meal and fermenting the mixed fermentation mixture with the coffee meal at 20° C. to 40° C. for 20 days to 40 days to prepare a coffee meal fermented solution, and aging the coffee meal fermented solution for 10 days to 1 month.

According to an exemplary embodiment, a microbial fermentation broth is produced by the method of producing the microbial fermentation broth.

According to an exemplary embodiment, a method of producing a deodorant includes filtering a by-product in the microbial fermentation broth to extract supernatant.

According to an exemplary embodiment, a deodorant is produced by the method of producing the deodorant.

The deodorant according to an embodiment of inventive concept includes one or more microorganisms selected from the group consisting of Lactobacillus parabuchneri, Lactobacillus casei, Lactobacillus buchneri, Bacillus subtilis, and Lactobacillus velezensis.

According to an exemplary embodiment, a deodorization method includes treating the deodorant on an object to be deodorized.

According to an exemplary embodiment, a method of producing an odor removing agent includes drying the by-product in the method of producing the deodorant.

According to an exemplary embodiment, an odor removing agent produced by the method of producing the odor removing agent.

The odor removing agent according to an embodiment of inventive concept includes one or more of Lactobacillus, Bacillus subtilis, and yeast fungus.

The odor removing agent according to an embodiment of inventive concept includes one or more of Aspergillus flavus and Aspergillus oryzae.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a flowchart illustrating a method of producing a microbial fermentation broth according to an embodiment of the inventive concept;

FIG. 2 is a flowchart illustrating a method of producing a deodorant and an odor removing agent according to an embodiment of the inventive concept;

FIG. 3 is a graph illustrating a deodorization effect on ammonia of the deodorant prepared in Example 2 over time; and

FIG. 4 is a graph illustrating a deodorization effect on trimethylamine of the deodorant prepared in Example 2 over time.

### DETAILED DESCRIPTION

Advantages and features of the inventive concept, and methods for achieving them will be apparent with reference to the embodiments described below in detail with the accompanying drawings. However, the inventive concept is not limited to the embodiments disclosed below, but will be implemented in various different forms. The embodiments make the disclosure of the inventive concept complete and provide to fully inform those skilled in the art to the scope of the invention. The inventive concept is defined by the scope of the claims.

In general, a conventional deodorant for removing an odor is the majority of a product to which a chemical component is added. When using a product including a harmful substance such as a heavy metal, there is a risk of causing secondary environmental pollution.

However, a microbial fermentation broth according to an embodiment of the inventive concept includes a microorganism cultured by fermenting coffee meal, which is a by-product of coffee made by pouring hot water onto ground coffee beans and allowing to be brewed. That is, as the microbial fermentation broth of the inventive concept includes eco-friendly materials without harmful substances, an extract of the microbial fermentation broth may be used to produce a deodorant and a by-product of the microbial fermentation broth may also be used as an odor removing agent of food waste or livestock manure. However, the microbial fermentation broth of the inventive concept may be not limited to intended use thereof and may be used in various fields.

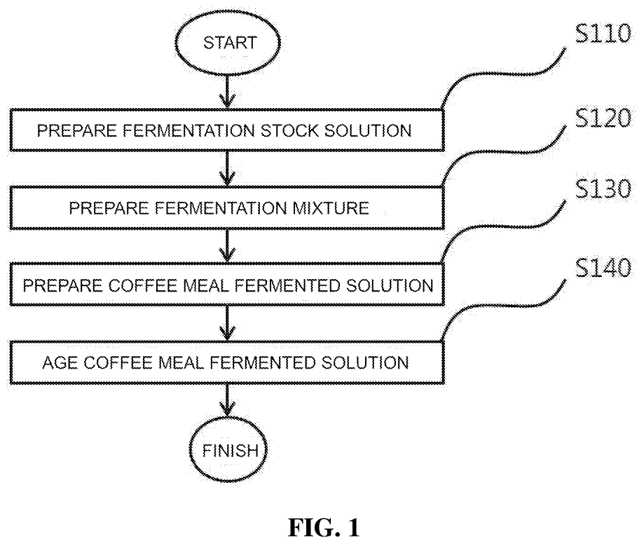

Hereinafter, referring to FIG. 1, a method of producing a microbial fermentation broth according to an embodiment of the inventive concept will be described step by step.

FIG. 1 is a flowchart illustrating a method of producing a microbial fermentation broth according to an embodiment of the inventive concept.

Referring to FIG. 1, the method of producing the microbial fermentation broth according to an embodiment of the inventive concept includes preparing a fermentation stock solution in S110, preparing a fermentation mixture in S120, preparing a coffee meal fermented solution in S130, and aging the coffee meal fermented solution in S140.

According to an embodiment of the inventive concept, in the preparing of the fermentation stock solution of S110, at least two of plum, dandelion, pine needle, and thistle are fermented at 20° C. to 40° C. for 4 months to 8 months to prepare each fermentation stock solution.

According to an embodiment of the inventive concept, S110 is not limited thereto, and the preparing of the fermentation stock solution may be performed for 5 months to 7 months and may be performed for about 6 months.

According to an embodiment of the inventive concept, the preparing of the fermentation stock solution may also be carried out under a temperature of 23° C. to 37° C. or 26° C. to 34° C.

According to an embodiment of the inventive concept, in the preparing of the fermentation mixture of S120, the fermentation stock solutions are mixed and the mixed fermentation stock solutions are fermented at 20° C. to 30° C. for 1 month to 5 months to prepare the fermentation mixture

According to an embodiment of the inventive concept, S120 is not limited thereto, and the preparing of the fermentation mixture' may be performed for 2 months to 4 months or may be performed for about 3 months. The preparing of the fermentation mixture may also be carried out under a temperature of 22° C. to 28° C. or 24° C. to 26° C.

According to an embodiment of the inventive concept, in the preparing of the coffee meal fermented solution of S130, the fermentation mixture and the coffee meal are mixed at 20° C. to 40° C. for 20 days to 40 days to prepare the coffee meal fermented solution.

According to an embodiment of the inventive concept, S130 is not limited thereto, and the preparing of the coffee meal fermented solution may be performed for 24 days to 36 days or may be performed for 28 days to 32 days.

According to an embodiment of the inventive concept, the preparing of the coffee meal fermented solution may also be carried out under a temperature of 23° C. to 37° C. or 26° C. to 34° C.

According to an embodiment of the inventive concept, in the aging of the coffee meal fermented solution of S140, the coffee meal fermented solution is aged for 10 days to 1 month to prepare the microbial fermentation broth. According to an embodiment of the inventive concept, S140 is not limited thereto, and the aging of the coffee meal fermented solution may be performed for 14 days to 26 days or 18 days to 22 days.

Subsequently, a method of producing a deodorant and an odor removing agent from the microbial fermentation broth according to an embodiment of the inventive concept will be described.

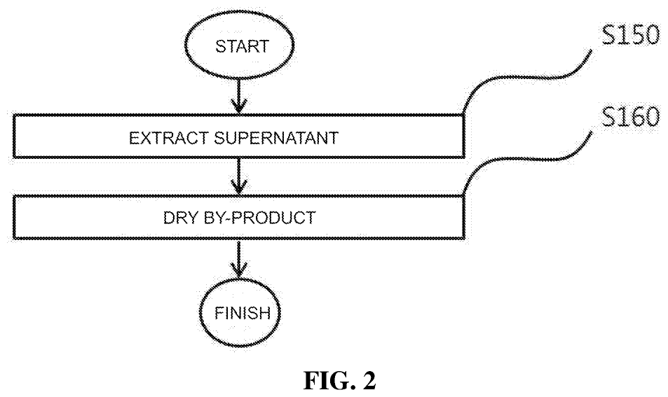

FIG. 2 is a flowchart illustrating a method of producing a deodorant and an odor removing agent according to an embodiment of the inventive concept.

The method of producing the deodorant includes extracting supernatant in S150.

Referring to FIG. 2, in the extracting of the supernatant of S150, the by-product is filtered from the microbial fermentation broth to extract the supernatant.

The deodorant including the supernatant extracted through the extracting of the supernatant in S150 may be produced. For example, a deodorant may be prepared by adding another material to the supernatant. Furthermore, a deodorant may be prepared by diluting a specific amount of water in the supernatant and a mixing ratio of the extracted supernatant and water may be adjusted to 9:1 to 1:9. In addition, the extracted supernatant may be directly sprayed and used as the deodorant, but is not limited thereto.

The deodorant according to an embodiment of the inventive concept may include one or more microorganisms selected from the group consisting of Lactobacillus parabuchneri, Lactobacillus casei, Lactobacillus buchneri, Bacillus subtilis, and Lactobacillus velezensis.

The microorganism is the most important substance in removing an odor gas and the deodorant including the microorganism is particularly effective in removing the odor floating in the air. When the deodorant is sprayed on the livestock manure or food waste, the odor is easily removed. In addition, a fungus may occur to be utilized in a production of eco-friendly compost. Detailed effects related to the above-described effects will be described later through embodiments of the inventive concept.

Referring to FIG. 2, the method of producing the odor removing agent according to an embodiment of the inventive concept includes drying the by-product in S160.

The drying of the by-product of S160 includes drying the remaining by-product after extracting the supernatant from the microbial fermentation broth in S150 to produce the odor removing agent. The drying may be preferably performed at 40° C. to 80° C., but is not limited thereto.

According to an embodiment of the inventive concept, the dried odor removing agent may be produced in a solid power form, and may include the following microorganisms.

According to an embodiment of the inventive concept, the odor removing agent may include one or more microorganisms of Aspergillus flavus and Aspergillus duck.

As the odor removing agent of the inventive concept includes the microorganisms as described above, when the solid odor removing agent is added to the food waste, a cause of the odor may be decomposed to remove the odor. In addition, it is effective to decompose salt and oil of the food waste and to promote the production of the fungus, thereby transforming the food waste into a resource.

Furthermore, when the odor removing agent of the inventive concept is dried and then is sprayed on the soil, the microorganisms and fungi turn the soil into a good state for plants to grow, thereby improving growth of crops.

In addition, the compost may be prepared by adding the odor removing agent to a raw material for compost. The compost raw material is referred to as a general raw material used in production of compost and the livestock manure may be used as the compost raw material, but is not limited thereto. When the odor removing agent prepared by the above-described method is mixed with the livestock manure, production of ammonia and trimethylamine may be significantly reduced.

Hereafter, it will be described with Examples to help understanding of the inventive concept. The following Examples are merely provided to more easily understand the inventive concept, but the contents of the inventive concept are not limited by Examples.

### EXAMPLE 1

### Production of Microbial Fermentation Broth

In an embodiment of the inventive concept, a microbial fermentation broth was prepared by the following method.

Raw materials of plum, dandelion, pine needles, thistles were put in a jar and first fermented for 6 months at 30° C., respectively. Fermentation stock solutions were extracted from fermented raw materials, respectively. The extracted fermentation stock solutions were mixed with one another to prepare a mixed solution and the mixed solution was second fermented at 24° C. for 3 months to prepare a fermentation mixture. Coffee meal such as coffee waste was added to the fermentation mixture to prepare a coffee meal mixture. The coffee meal mixture was third fermented at 30° C. for 30 days to prepare a coffee meal fermented solution. The coffee meal fermented solution was further aged for 15 days to prepare the microbial fermentation broth. Here, an aging temperature was performed at room temperature.

### EXAMPLE 2

### Production of Deodorant

The deodorant was prepared from the microbial fermentation broth prepared in Example 1.

Clear supernatant which was produced on a top of the microbial fermentation broth was extracted. The obtained supernatant and water were mixed at a ratio of 3:1 to prepare the deodorant.

As a result of odor removal sensory evaluation, it was shown that the deodorant prepared by the above-described method had an excellent odor removal effect in comparison with Comparative Example without the deodorant (see Example 4).

### EXAMPLE 3

### Deodorization Method

The inventive concept provides a deodorization method including treating the deodorant to an object to be deodorized.

The deodorization method may include diluting the deodorant in water and spraying the diluted deodorant as needed. The deodorant may be sprayed directly onto odor generating waste, such as food waste, to remove the odors. In addition, the deodorant may be sprayed in the air at or around an odor generating region, but is not limited thereto.

In addition, the deodorant may be used directly to livestock, or may be used for manure of livestock. The livestock may be chickens, goats, pigs, cattle, cows, horses, donkeys, sheep, dogs, rabbits, cats, ducks, geese, pheasant, quail, ostrich, mallard, turkey, or the like, but is not limited thereto.

### EXAMPLE 4

### Confirmation of Deodorization Effect of Deodorant

1. Test Method

Test organization: Korea Conformity Laboratories (KCL)

Test environment: (22.5±0.6)° C., (42.1±0.8)% R. H.

Test equipment: gas detector having a detecting tube type (GV-100S, Gastec, Japan)

Detection limit: 0.2 μmol/mol

Test item: ammonia, trimethylamine

The deodorization effect of the deodorant prepared in Example 2 was tested through sensory evaluation. The measuring method is as follows.

First, 20 g of the deodorant of the inventive concept was placed in a 5 L reactor and sealed, a test gas having an initial concentration of 50 μmol/mol was injected, and gas concentrations were measured every 30 minutes to obtain sample concentrations. Subsequently, a blank concentration was obtained by performing the above-described test without injecting the deodorant of the inventive concept.

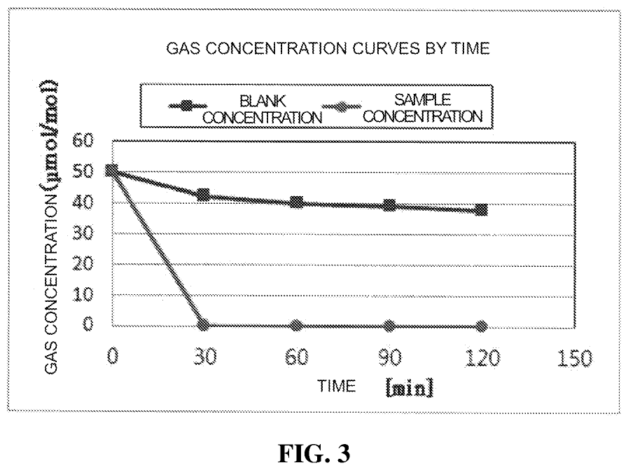

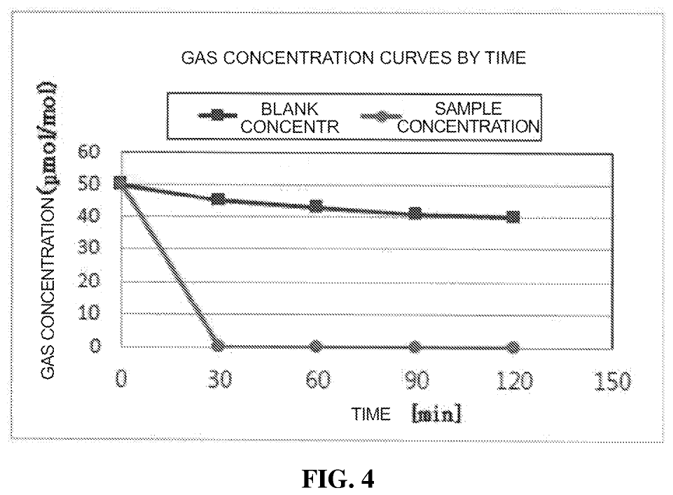

The concentrations of the test gas were measured by a gas detector tube (SPS-KCLI2218-6218). The measurement results obtained were shown in Tables 1 and 2 and FIGS. 3 and 4. A reduction rate of test gas concentrations by time was calculated by the following equation.

$$Reduction rate of test gas concentration ( \% ) = \frac{\{ \begin{matrix}( blank concentration ) - \\ ( sample concentration )\end{matrix} \}}{( blank concentration )} \times 100 \% \qquad [ Equation 1 ]$$

2. Test Result

Hereinafter, for the convenience of description, the blank concentration and the sample concentration obtained through the above-described test will be referred to as Comparative Example and Example, respectively.

TABLE 1

| | | | Test result | | |
|---|---|---|---|---|---|
| | | | Blank | Sample | Concentration |
| | | | concentration | concentration | reduction rate |
| Test item | | Unit | (μmol/mol) | (μmol/mol) | (%) |
| Ammonia | 0 min | % | 50 | 50 | 0.0 |
| NH₃ | 30 min | % | 42 | <0.2 | 99.5 |
| | 60 min | % | 40 | <0.2 | 99.5 |
| | 90 min | % | 39 | <0.2 | 99.5 |
| | 120 min | % | 38 | <0.2 | 99.5 |

FIG. 3 is a graph illustrating a deodorization effect on ammonia of the deodorant prepared in Example 2 over time. Referring to FIGS. 1 and 3, it was seen that the ammonia concentration of the test item was significantly reduced in Example over time in comparison to Comparative Example.

TABLE 2

| | | | Test result | | |
|---|---|---|---|---|---|
| | | | Blank | Sample | Concentration |
| | | | concentration | concentration | reduction rate |
| Test item | | Unit | (μmol/mol) | (μmol/mol) | (%) |
| Trimethyl- | 0 min | % | 50 | 50 | 0.0 |
| amine | 30 min | % | 45 | <0.2 | 99.6 |
| (CH₃)₃N | 60 min | % | 43 | <0.2 | 99.5 |
| | 90 min | % | 41 | <0.2 | 99.5 |
| | 120 min | % | 40 | <0.2 | 99.5 |

FIG. 4 is a graph illustrating a deodorization effect on trimethylamine of the deodorant prepared in Example 2 over time. Referring to FIGS. 2 and 4, it was seen that the trimethylamine concentration of the test item was significantly reduced in Example over time in comparison to Comparative Example.

As described above, as the results of measuring the concentration of the test items over time, it was seen that both of the concentrations of ammonia and trimethylamine were removed 99% or more in Example where the deodorant of the inventive concept is injected. Therefore, it was confirmed that the deodorant of the inventive concept sufficiently fulfills a deodorization role.

### EXAMPLE 5

### Microorganism Identification of Deodorant

To identify the microorganisms included in the deodorant prepared in Example 2, separation and identification test of microorganisms was carried out using a ribosomal RNA (rRNA) sequence analysis method in Chungnam National University Agricultural Science Research Institute.

1. Test Method

In Example 1, the deodorant was centrifuged to isolate strains for each raw material. Sequencing of each isolated strain was performed to obtain base sequences. The obtained base sequences were compared with base sequences in database of the National Center for Biotechnology Information (NCBI) to analyze species with high similarity.

2. Test Result

As a result of analyzing the strains, it was identified that the strains include one or more microorganisms selected from the group consisting of Lactobacillus parabuchneri, Lactobacillus casei, Lactobacillus buchneri, Lactobacillus subtilis, and Lactobacillus velezensis.

TABLE 3

| Test item | Unit | Test result |
|---|---|---|
| Lactobacillus Parabuchneri | CFU/ml | 1.6 × 10⁴ |
| Lactobacillus Casei | CFU/ml | 2.0 × 10⁴ |
| Lactobacillus Buchneri | CFU/ml | 1.3 × 10⁴ |
| Lactobacillus Subtilis | CFU/ml | 1.8 × 10⁴ |
| Lactobacillus Velezensis | CFU/ml | 1.1 × 10⁴ |

### EXAMPLE 6

### Microorganism Identification of Odor Removing Agent

To separate the microorganisms of the odor removing agent obtained by drying the remaining by-product after preparing the deodorant of Example 2, separation and identification test of microorganisms was carried out using a ribosomal RNA (rRNA) sequence analysis method in Chungnam National University Agricultural Science Research Institute.

1. Test method

1) Gemonic DNA Extraction

5% Boiling Resin of 150 μl+Pro-K (20 ng/ml) of 10 μl were prepared, an appropriate amount of cells were taken and dissolved therein, and then incubated at 60° C. for 30 minutes. Thereafter, the above-incubated mixture was boiled at 100° C. for 10 minutes and left in an ice bath for 2 minutes, and this process was repeated three times. Subsequently, 3 μl of the supernatant was extracted by centrifugation at 13,000 rpm for 10 minutes.

2) PCR Amplification and Purification

PCR was carried out using a template for the extracted supernatant.

3) Sequencing

Sequencing of 18S rRNA portion of each strain was performed. A base sequence obtained through the sequencing is shown in SEQ ID NO. 1. The obtained base sequences were compared with base sequences in database of the NCBI to analyze species with high similarity.

2. Test Result

As a result of analyzing detected bacteria, it was identified that the bacteria include Lactobacillus, Bacillus subtilis, and yeast fungus as follows.

TABLE 4

| Test item | Unit | Test result |
|---|---|---|
| lactobacillus | CFU/ml | 7.0 × 10⁶ |
| bacillus subtilis | CFU/ml | 3.4 × 10⁶ |
| yeast fungus | CFU/ml | 3.0 × 10⁵ |

As a result of analyzing the strains, it was identified that the strains include one or more selected from the group consisting of Aspergillus flavus and Aspergillus oryzae.

TABLE 5

| Test item | Unit | Test result |
|---|---|---|
| Aspergillus flavus | CFU/ml | 4.0 × 10⁵ |

### EXAMPLE 7

### Heavy Metal Detection Test

The heavy metal detection test was performed for the deodorant prepared in Example 2.

Test organization: Korea Testing and Research (KTR)

Test environment: temperature (20±5)° C., humidity (40±20)%

Test item: Pb, Cd, Hg, Cr(Vl)

TABLE 6

| Test item | Unit | Test Result | Detection Limit |
|---|---|---|---|
| Pb | mg/kg | Not detected | 5 |
| Cd | mg/kg | Not detected | 0.5 |
| Hg | mg/kg | Not detected | 0.5 |
| Cr(Vl) | mg/kg | Not detected | 0.5 |

As described in Table 3, as results of the detection test for Pb, Cd, Hg, Cr(Vl), it was seen that all of the substances in the deodorant were undetected. That is, it was seen that the deodorant of the inventive concept was an eco-friendly material without heavy metals.

### EXAMPLE 8

### Hazardous Substance Detection Test

The hazardous substance detection test was conducted on the deodorant prepared in Example 2.

Test organization: Korea Conformity Laboratories (KCL)

TABLE 7

| Test item | Unit | Test Result |
|---|---|---|
| formaldehyde | mg/kg | Not detected (Detection Limit 5) |
| acetaldehyde | mg/kg | Not detected (Detection Limit 2) |
| methanol | mg/kg | Not detected (Detection Limit 100) |
| naphthalene | mg/kg | Not detected (Detection Limit 1) |
| nickel | mg/kg | 0.27 |
| chloroform | mg/kg | Not detected (Detection Limit 1) |

Referring to Table 4, in a case of nickel, 0.27 mg/kg was detected. It was confirmed to correspond to a very small amount below a worrisome level of soil contamination of nickel of 100 mg/kg, which is noticed by the Ministry of Environment. Therefore, it was seen that the deodorant of the inventive concept meets safety and labeling standards on a risk concerned product (Ministry of Environment Notice 2016-254, Dec. 30, 2016) through the above-described test.

### EXAMPLE 9

### Environmental Certification

The deodorant prepared in Example 2 was tested for compliance with certification criteria of an eco-label product.

Test organization: Korea Conformity Laboratories (KCL)

Test environment: temperature (20 to 30)° C., humidity (40 to 60)% R. H.

Test result: pass

The deodorant of the inventive concept was confirmed to meet the certification criteria of the eco-label product according to Article 17 (3) of the Environmental Technology and Environmental Industry Support Act, Article 23 (2) of the Enforcement Decree of the Act, and Article 34 (2) of the Enforcement Regulation of the Act, to obtain an eco-label certificate.

As described above, in the microbial fermentation broth prepared through the method of producing the microbial fermentation broth of the inventive concept, the microorganisms are cultured in the producing process and it is effective for deodorization.

In addition, the deodorant may be produced using the extract of the microbial fermentation broth and the by-product may be used as the odor removing agent. Therefore, waste is not produced, and thus economical effect is obtained.

Furthermore, as the microbial fermentation broth of the inventive concept is identified as the eco-friendly material without the hazardous substances, such as the heavy metals, there is no secondary environmental damage in use.

Accordingly, when the deodorant and odor removing agent produced by the microbial fermentation broth of the inventive concept are spread to the farmhouse, income of the farmhouse may be increased and civil complaints caused by the odor may be solved.

According to the method of producing the microbial fermentation broth of the inventive concept, the extract of microbial fermentation broth may remove the odors and the by-product may be used as compost

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method of producing a microbial fermentation broth, the method comprising:
fermenting at least two of plum, dandelion, pine needle, and thistle at 20° C. to 40° C. for 4 months to 8 months to prepare each fermentation stock solution;
mixing the fermentation stock solutions and fermenting the mixed fermentation stock solutions at 20° C. to 30° C. for 1 month to 5 months to prepare a fermentation mixture;
mixing the fermentation mixture with coffee meal and fermenting the mixed fermentation mixture with the coffee meal at 20° C. to 40° C. for 20 days to 40 days to prepare a coffee meal fermented solution; and
aging the coffee meal fermented solution for 10 days to 1 month.

2. A microbial fermentation broth produced by the method of claim 1.

3. A method of producing a deodorant, the method comprising:
filtering a by-product in the microbial fermentation broth of claim 2 to extract supernatant.

4. A deodorant produced by the method of claim 3.

5. The deodorant of claim 4, wherein the deodorant includes one or more microorganisms selected from the group consisting of Lactobacillus parabuchneri, Lactobacillus casei, Lactobacillus buchneri, Bacillus subtilis, and Lactobacillus velezensis.

6. A deodorization method comprising:
treating the deodorant of claim 4 on an object to be deodorized.

7. A method of producing an odor removing agent, the method comprising:
drying the by-product in the method of claim 3.

8. An odor removing agent produced by the method of claim 7.

9. The odor removing agent of claim 8, wherein the odor removing agent includes one or more of Lactobacillus, Bacillus subtilis, and yeast fungus.

10. The odor removing agent of claim 8, wherein the odor removing agent includes one or more of Aspergillus flavus and Aspergillus oryzae.


SEQUENCE LISTING (1 sequence)

SEQ ID NO: 1 - DNA, 591 bp, Artificial Sequence
  synthesized
aacaaggtttccgtaggtgaacctgcggaaggatcattaccgagtgtagggttcctagcgagcccaacctcccacccgtgtttactgtaccttagttgcttcggcgggcccgccattcatggccgccgggggctctcagccccgggcccgcgcccgccggagacaccacgaactctgtctgatctagtgaagtctgagttgattgtatcgcaatcagttaaaactttcaacaatggatctcttggttccggcatcgatgaagaacgcagcgaaatgcgataactagtgtgaattgcagaattccgtgaatcatcgagtctttgaacgcacattgcgccccctggtattccggggggcatgcctgtccgagcgtcattgctgcccatcaagcacggcttgtgtgttgggtcgtcgtcccctctccgggggggacgggccccaaaggcagcggcggcaccgcgtccgatcctcgagcgtatggggctttgtcacccgctctgtaggcccggccggcgcttgccgaacgcaaatcaatctttccaggttgacctcggatcaggtagggatacccgctgaacttaagcatatcaa